(19) United States
(12) Patent Application Publication
(10) Pub. No.: US 2013/0045189 A1
(43) Pub. Date: Feb. 21, 2013

(54) METHODS OF TREATING STROKE THROUGH ADMINISTRATION OF CTX0E03 CELLS

(71) Applicants: UNIVERSITY OF SOUTH FLORIDA, Tampa, FL (US); RENEURON LIMITED, Surrey (GB)

(72) Inventors: Paul R. Sanberg, Spring Hill, FL (US); John Sinden, Surrey (GB)

(73) Assignees: RENEURON LIMITED, Surrey (GB); UNIVERSITY OF SOUTH FLORIDA, Tampa, FL (US)

(21) Appl. No.: 13/660,647

(22) Filed: Oct. 25, 2012

Related U.S. Application Data
(63) Continuation of application No. PCT/US2011/033945, filed on Apr. 26, 2011.
(60) Provisional application No. 61/327,967, filed on Apr. 26, 2010.

(51) Int. Cl. A61K 35/30; A61P 25/00; A61P 25/28

(52) U.S. Cl. 424/93.7

(57) ABSTRACT

The subject invention pertains to methods to enhance the therapeutic effects of cellular or drug treatment in various diseases and disorders. More particularly, the present invention provides methods of treating disorders by administering CTX0E03 cells to the patient, intravenously or intraarterially. The treatment is useful for neurodegenerative diseases, such as stroke. The CTX0E03 cells may be cryopreserved and/or passaged before administration into the patient. Administration of the CTX0E03 cells into stroke rat models was at or within 48 hours after stroke. Testing of the rat models through elevated body swing test to measure of neurobehavioral status at the time of transplant and repeated triphenyltetrazolium chloride (TTC) staining as a measure of infarct volume showed short term survival that provided significant protection from the stroke.

### CROSS REFERENCE TO RELATED APPLICATIONS

The application is a continuation of and claims priority to prior filed International Application No. PCT/US2011/033945, entitled “Methods of Treating Stroke Through Administration of CTX0E03 Cells” filed Apr. 26, 2011, which claims priority to U.S. Provisional Patent Application No. 61/327,967, entitled, “Intravascular Administration of CTX0E03 Stem Cells Exerts Benefit in Acute Stroke Animals”, filed 26 Apr., 2010, the contents of which are herein incorporated by reference.

### FIELD OF INVENTION

This invention relates to the treatment of various neural diseases and disorders using stem cells. Specifically, the invention provides administering the conditionally immortalized fetal neural stem cell line CTX0E03 to treat stroke.

### BACKGROUND OF THE INVENTION

Cerebrovascular disease, considered one of the top five non-communicable diseases, affects approximately 50 million people worldwide, resulting in approximately 5.5 million deaths per year. Of those 50 million, stroke accounts for roughly 40 million people. Stroke is the third leading cause of death in developed countries and accounts for the major cause of adult disability.

Despite the significant research into stroke, there are depressingly few effective treatments for acute stroke, with organized stroke care, early aspirin and thrombolytic treatment being the only proven therapeutic strategies (Dawson & Walters (2006). New and emerging treatments for stroke. Br Med Bull. 77-78). Infarct volume increases in the first few hours after onset of ischaemic stroke, with the infarct gradually subsuming the ischaemic penumbra, the region where blood supply is significantly reduced but energy metabolism is maintained because of collateral flow. Survival of neurons in the penumbra depends on the severity and duration of ischaemia, however prior to reperfusion a physiological cascade occurs, which increases intracellular calcium (Dawson & Walters (2006). New and emerging treatments for stroke. Br Med Bull. 77-78). This cascade self-perpetuates causing acidosis, activation of lipase, protease and free radical generation (Dawson & Walters (2006). New and emerging treatments for stroke. Br Med Bull. 77-78).

For ischaemic and haemorrhagic stroke there are therapeutic targets which exist only in the early hours after stroke, requiring rapid assessment and treatment. However, studies found that only 30% of those suspected stroke patients received a CT or other scan on the same day.

Stroke treatment consists of two categories: prevention and acute management. Prevention treatments currently consist of antiplatelet agents, anticoagulation agents, surgical therapy, angioplasty, lifestyle adjustments, and medical adjustments. An antiplatelet agent commonly used is aspirin. The use of anticoagulation agents seems to have no statistical significance. Surgical therapy appears to be effective for specific sub-groups. Angioplasty is still an experimental procedure with insufficient data for analysis. Lifestyle adjustments include quitting smoking, regular exercise, regulation of eating, limiting sodium intake, and moderating alcohol consumption. Medical adjustments include medications to lower blood pressure, lowering cholesterol, controlling diabetes, and controlling circulation problems.

Acute management treatments consist of the use of thrombolytics, neuroprotective agents, Oxygenated Fluorocarbon Nutrient Emulsion (OFNE) Therapy, Neuroperfusion, GPIIb/IIIa Platelet Inhibitor Therapy, and Rehabilitation/Physical Therapy.

A thrombolytic agent induces or moderates thrombolysis, and the most commonly used agent is tissue plasminogen activator (t-PA). Recombinant t-PA (rt-PA) helps reestablish cerebral circulation by dissolving (lysing) the clots that obstruct blood flow. It is an effective treatment, with an extremely short therapeutic window; it must be administered within 3 hours from onset. It also requires a CT scan prior to administration of the treatment, further reducing the amount of time available. Genetech Pharmaceuticals manufactures ACTIVASE® and is currently the only source of rt-PA. Recent studies have found that the odds of favourable outcome were 2.8 (95% CI=1.8-9.5) if tPA is administered within 90 min and 1.6 (95% CI=1.1-2.2) between 91 and 180 min, showing that the chances of being free of handicap after stroke are increased nearly 3-fold by thrombolytic treatment, provided it is administered within 90 min of onset (Dawson & Walters (2006). New and emerging treatments for stroke. Br Med Bull. 77-78).

Neuroprotective agents are drugs that minimize the effects of the ischemic cascade, and include, for example, Glutamate Antagonists, Calcium Antagonists, Opiate Antagonists, GABA-A Agonists, Calpain Inhibitors, Kinase Inhibitors, and Antioxidants. Several different clinical trials for acute ischemic stroke are in progress. Due to their complementary functions of clot-busting and brain-protection, future acute treatment procedures will most likely involve the combination of thrombolytic and neuroprotective therapies. However, like thrombolytics, most neuroprotectives need to be administered within 6 hours after a stroke to be effective.

Oxygenated Fluorocarbon Nutrient Emulsion (OFNE) Therapy delivers oxygen and nutrients to the brain through the cerebral spinal fluid. Neuroperfusion is an experimental procedure in which oxygen-rich blood is rerouted through the brain as a way to minimize the damage of an ischemic stroke. GPIIb/IIIa Platelet Inhibitor Therapy inhibits the ability of the glycoprotein GPIIb/IIIa receptors on platelets to aggregate, or clump. Rehabilitation/Physical Therapy must begin early after stroke, however, they cannot change the brain damage. The goal of rehabilitation is to improve function so that the stroke survivor can become as independent as possible.

Although some of the acute treatments showed promise in clinical trials, a study conducted in Cleveland showed that only 1.8% of patients presenting with stroke symptoms even received the t-PA treatment (Katzan I L, et al. (2000) Use of tissue-type plasminogen activator for acute ischemic stroke: the Cleveland area experience. JAMA, 283:1151-1158). t-PA is currently the most widely used of the above-mentioned acute stroke treatments, however, the number of patients receiving any new “effective” acute stroke treatment is estimated to be under 10%. These statistics show a clear need for the availability of acute stroke treatment at greater than 24 hours post stroke.

For some of these acute treatments (i.e., t-PA) the time of administration is crucial. Recent studies have found that 42% of stroke patients wait as long as 24 hours before arriving at the hospital, with the average time of arrival being 13 hours after stroke. t-PA has been shown to enhance recovery of ˜⅓ of the patients that receive the therapy, however a recent study mandated by the FDA (Albers, et al. (2000). Intravenous Tissue-Type Plasminogen Activator for Treatment of Acute Stroke, The Standard Treatment with Alteplase to Reverse Stroke Study. JAMA. 283(9)) found that about a third of the time the three-hour treatment window was violated resulting in an ineffective treatment. With the exception of rehabilitation, the remaining acute treatments are still in clinical trials and are not widely available in the U.S., particularly in rural areas, which may not have large medical centers with the needed neurology specialists and emergency room staffing, access to any of these new methods of stroke diagnosis and therapy may be limited for some time.

The cost of stroke in the US is over $43 billion, including both direct and indirect costs. The direct costs account for about 60% of the total amount and include hospital stays, physicians' fees, and rehabilitation. These costs normally reach $15,000/patient in the first three months; however, in approximately 10% of the cases, the costs are in excess of $35,000. Indirect costs account for the remaining portion and include lost productivity of the stroke victim, and lost productivity of family member caregivers (National Institute of Neurological Disorders and Stroke, National Institutes of Health, Bethesda, Md.).

Approximately 750,000 strokes occur in the US every year, of which about ⅓ are fatal. Of the remaining patients, approximately ⅓ is impaired mildly, ⅓ is impaired moderately, and ⅓ is impaired severely. Ischemic stroke accounts for 80% of these strokes.

As the baby-boomers age, the total number of strokes is projected to increase substantially. The risk of stroke increases with age. After age 55, the risk of having a stroke doubles every decade, with approximately 40% of individuals in their 80's having strokes. Also, the risk of having a second stroke increases over time. The risk of having a second stroke is 25-40% five years after the first. With the over-65 portion of the population expected to increase as the baby boomers reach their golden years, the size of this market will grow substantially. Also, the demand for an effective treatment will increase dramatically.

Given the inability to effectively mitigate the devastating effects of stroke, it is imperative that novel therapeutic strategies are developed to both minimize the initial neural trauma as well as repair the damage brain once the pathological cascade of stroke has run its course.

Transplantation of stem cells has been proposed as a means of treating stroke. Neural stem cells are important treatment candidates for stroke and other CNS diseases because of their ability to differentiate in vitro and in vivo into neurons, astrocytes and oligodendrocytes. The powerful multipotent potential of stem cells may make it possible to effectively treat diseases or injuries with complicated disruptions in neural circuitry, such as stroke where more than one cell population is affected. Human umbilical cord blood (HUCB) cells administered systemically is a very effective treatment for experimental stroke in rats. However, HUCB cellular therapy is limited for translation as a treatment for humans because of the potential for disease and availability of these cells.

Because of the difficulty in effectively treating patients after stroke, there is a need in the art for methods to enhance the treatment of stroke.

Stem cell implants have shown some degree of success in the middle cerebral artery occlusion (MCAO) rodent model of stroke. Previous work has demonstrated that a single intravenous injection of human umbilical cord blood cell (hUCBC) in aged rats can significantly reduce the number of activated microglia, and increase neurogenesis, thus improving the microenvironment of the aged hippocamcus and rejuvenating the aged neural stem/progenitor cells (Bachstetter A D, et al. (2008). Peripheral injection of human umbilical cord blood stimulates neurogenesis in the aged rat brain. BMC Neurosci. 9:22). However, hUCBC are limited by the number of cells attainable from cord collection, which limits the effectiveness of such a treatment. Further, it has been observed that cells (hES-NPC) administered into the blood stream were only capable of migrating into the CNS at less than 1% the total cells administered (Crokcer, et al. (2011). Intravenous administration of human ES-derived neural precursor cells attenuates cuprizone-induced CNS demyelination. Neuropathol Appl Neurobiol. doi: 10.1111/j.1365-2990.2011.01165.x. [Epub ahead of print]).

Several studies have shown growth factors are vital in responding to ischemia-induced brain damage by enhancing the survival, stimulating the proliferation of endogenous neural progenitor cells, and initiating differentiation of those progenitor cells (Kalluri, et al. (2008). Growth factors, stem cells, and stroke. Neurosurg Focus. 24(3-4)). For example, growth factors such as TGFβ and BMP-413 can promote the differentiation of neural stem cells, and BMP-4 can promote the differentiation of smooth-muscle cells and glial cells (Kalluri, et al. (2008). Growth factors, stem cells, and stroke. Neurosurg Focus. 24(3-4):E14). IGF-I can stimulate the proliferation of progenitor cells when in the presence of mitogens like FGF-2 and promotes differentiation after FGF-2 withdrawal (Yamashita, et al. (2009) Gene and stem cell therapy in ischemic stroke. Cell Transplant. 18(9); 999-1002. Epub 2009 Apr. 29). MCP-1, erythropoietin, and MMPs are involved in the migration of neuroblasts to the site of injury (Yamashita Yamashita, et al. (2009) Gene and stem cell therapy in ischemic stroke. Cell Transplant. 18(9); 999-1002. Epub 2009 Apr. 29). GDNF was also found to reduce infarct size and brain edema after topical application (Yamashita, et al. (2009) Gene and stem cell therapy in ischemic stroke. Cell Transplant. 18(9); 999-1002. Epub 2009 Apr. 29).

However, current progenitor cell treatments rely on transplant of cells into the brain, which is an invasive and dangerous surgery. What is needed is a simple, safe method of administering neural progenitor cells into a patient after stroke or other neurodegenerative disease onset.

### SUMMARY OF THE INVENTION

This invention is intended to overcome, or at least alleviate, one or more of the difficulties or deficiencies associated with the prior art. In that regard, the present invention provides methods to enhance the therapeutic effects of cellular or drug treatment in various diseases and disorders. Preferably, the disorder is stroke.

In that regard, the present invention fulfills in part the need to identify new, unique methods for treating strokes.

Accordingly, a method is provided for treating a neurodegenerative disease in a patient or repairing neural damage caused by a disease or disorder, by administering a therapeutically effective amount of CTX0E03 cells to the patient, where the administration is performed intravenously (IV) or intraarterially (IA). The treatment is useful for neurodegenerative diseases, such as stroke. The CTX0E03 cells may be administered at about 1.0×10⁴ cells to about 1.0×10⁹ cells, more specifically at about 1×10⁵ to about 1×10⁷ cells. In particular embodiments, the CTX0E03 cells are administered at 1×10⁷. The cells are optionally administered in a therapeutic composition, such as a composition comprising Hank's balanced salt solution and N-acetylcycsteine.

The CTX0E03 cells are optionally cryopreserved before use and may also, in some variations, also be passaged before administration into the patient. Administration of the CTX0E03 cells may be performed at any therapeutically effective time, however, it has been found that IV or IA administration of the CTX0E03 cells within 2 days of stroke, and more specifically at 48 hours after stroke, unexpectedly provides ischemic neurons with statistically significant protection from the stroke. The elevated body swing test (EBST) as a marker of motor asymmetry was used as a measure of neurobehavioral status at the time of transplant and repeated at 3 days after cell implantation along with triphenyltetrazolium chloride (TTC) staining as a measure of infarct volume. Short term survival was also studied as an indication of the safety of the cell transplantation.

### BRIEF DESCRIPTION OF THE DRAWINGS

For a fuller understanding of the invention, reference should be made to the following detailed description, taken in connection with the accompanying drawings, in which:

FIG. 1 is a graph showing significant decrease in death rate following cell transplant. Vehicle: n=8 before treatment and n=4 2 days after; Cells: n=4 before treatment and n=4 2 days after. *p<0.05 Chi-square test

FIG. 2 is a graph showing EBST as a measure of motor asymmetry in vehicle and cell-treated animals. Motor asymmetry was significantly reduced in the cell-treated animals but not the vehicle. N=4 for both groups. *p<0.05 two-tailed t-test compared with pre-treatment group.

FIG. 3 is a graph showing the comparison of mean infarct size between vehicle and cell treated animals. Non-significant difference between infarct size as determined by t-test. TTC staining of two comparable sections is shown. N=4 for both groups.

FIG. 4 is a graph showing significant correlation between infarct size and motor asymmetry, as measured by EBST, in cell-treated animals. N=4. (r²=0.87; p<0.05).

FIG. 5 is a graph showing BrdU staining of proliferating cells shown in the SGZ and SGZ/GCL, respectively, of vehicle implanted and CTX0E03-implanted rats 2 days after transplant. The differences between CTX0E03 and vehicle are significant for total BrdU-positive cell counts (P<0.001) in cell- and vehicle-implanted SGZ based on the optical fractionator method of unbiased stereological analysis. BrdU, bromodeoxyuridine; GCL, granular cell layer; SGZ, subgranular zone.

FIG. 6 is a graph showing DCX staining of proliferating cells shown in the SGZ and SGZ/GCL, respectively, of vehicle implanted and CTX0E03-implanted rats 2 days after transplant. The differences between CTX0E03 and vehicle are significant for total BrdU-positive cell counts (P<0.005) in cell- and vehicle-implanted SGZ based on the optical fractionator method of unbiased stereological analysis. DCX, doublecortin; GCL, granular cell layer; SGZ, subgranular zone.

FIG. 7 is an image showing colocalization of BrdU and DCX staining within the SGZ. Double labeling of cells for BrdU (dark gray) and DCX (light gray) shown in orthogonal projection following confocal imaging. BrdU, bromodeoxyuridine; DCX, doublecortin; SGZ, subgranular zone.

FIG. 8(A) through (C) are images showing the existence of CTX0E03 grafts in the ventricle, but not in the SGZ. Images (A) and (C) show a number of HuNu-positive cells (indicated by the arrows) present along the need tract (shown by the white-dotted line) and the ventricle. Few HuNu-positive cells colocalize with BrdU-positive cells (seen in B and dark gray structures in C). Abbreviations: BrdU, bromodeoxyuridine, HuNu, human nuclei antigen; SGZ, subgranular zone.

### DETAILED DESCRIPTION OF THE PREFERRED EMBODIMENT

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “neurodegenerative disease” is used herein to describe a disease which is caused by damage to the central nervous system and which damage can be reduced and/or alleviated through transplantation of neural cells according to the present invention to damaged areas of the brain and/or spinal cord of the patient. Exemplary neurodegenerative diseases which may be treated using the neural cells and methods according to the present invention include for example, Huntington's disease, amyotrophic lateral sclerosis (Lou Gehrig's disease), lysosomal storage disease (“white matter disease” or glial/demyelination disease, as described, for example by Folkerth R D. (1999). Abnormalities of developing white matter in lysosomal storage diseases. J Neuropathol Exp Neurol. 58(9):887-902. Review), multiple sclerosis, brain injury or trauma caused by ischemia, accidents, environmental insult, etc. In addition, the present invention may be used to reduce and/or eliminate the effects on the central nervous system of a stroke or a heart attack in a patient, which is otherwise caused by lack of blood flow or ischemia to a site in the brain of said patient or which has occurred from physical injury to the brain and/or spinal cord. Neurodegenerative diseases also include neurodevelopmental disorders including for example, autism and related neurological diseases such as schizophrenia, among numerous others.

The isolation, manufacture and protocols for the CTX0E03 cell line in generating cells in the present invention is described in detail by Sinden, et al. (U.S. Pat. No. 7,416,888). In one application of the cells, a clinical trial for the stereotactic intracerebral administration of CTX0E03 drug product for the treatment of stable motor disability, 6 months to 5 years after a stroke is underway in Glasgow, Scotland (ClinicalTrials.gov, National Institutes of Health, Identifier #NCT01151124).

The neural stem cells of the subject invention can be administered to patients, including veterinary (non-human animal) patients, to alleviate the symptoms of a variety of pathological conditions for which cell therapy is applicable. For example, the cells of the present invention can be administered to a patient to alleviate the symptoms of neurological disorders such as stroke (e.g., cerebral ischemia, hypoxia-ischemia); neurodegenerative diseases, such as Huntington's disease; traumatic brain injury; amyotrophic lateral sclerosis; multiple sclerosis (MS) and other demyelinating diseases. In a preferred embodiment of the present invention, the cells are administered to alleviate the symptoms of stroke.

The term “patient” is used herein to describe an animal, preferably a human, to whom treatment, including prophylactic treatment, with the cells according to the present invention, is provided. For treatment of those infections, conditions or disease states which are specific for a specific animal such as a human patient, the term patient refers to that specific animal. The term “donor” is used to describe an individual (animal, including a human) who or which donates umbilical cord blood or fetal neural stem cells for use in a patient.

The term “effective amount” is used herein to describe concentrations or amounts of components such as differentiation agents, fetal neural stem cells, precursor or progenitor cells, specialized cells, such as neural and/or neuronal or glial cells, blood brain barrier permeabilizers and/or other agents which are effective for producing an intended result including differentiating stem and/or progenitor cells into specialized cells, such as neural, neuronal and/or glial cells, or treating a neurological disorder or other pathologic condition including damage to the central nervous system of a patient, such as a stroke, heart attack, or accident victim or for effecting a transplantation of those cells within the patient to be treated. Compositions according to the present invention may be used to effect a transplantation of the fetal neural stem cells within the composition to produce a favorable change in the brain or spinal cord, or in the disease or condition treated, whether that change is an improvement (such as stopping or reversing the degeneration of a disease or condition, reducing a neurological deficit or improving a neurological response) or a complete cure of the disease or condition treated.

The terms “stem cell” or “progenitor cell” are used interchangeably herein to refer to umbilical cord blood-derived stem and progenitor cells. The terms stem cell and progenitor cell are known in the art (e.g., Stem Cells: Scientific Progress and Future Research Directions, report prepared by the National Institutes of Health, June, 2001). The term “neural cells” are cells having at least an indication of neuronal or glial phenotype, such as staining for one or more neuronal or glial markers or which will differentiate into cells exhibiting neuronal or glial markers. Examples of neuronal markers which may be used to identify neuronal cells according to the present invention include, for example, neuron-specific nuclear protein, tyrosine hydroxylase, microtubule associated protein, and calbindin, among others. The term neural cells also includes cells which are neural precursor cells, i.e., stem and/or progenitor cells which will differentiate into or become neural cells or cells which will ultimately exhibit neuronal or glial markers, such term including pluripotent stem and/or progenitor cells which ultimately differentiate into neuronal and/or glial cells. All of the above cells and their progeny are construed as neural cells for the purpose of the present invention. Neural stem cells are cells with the ability to proliferate, exhibit self-maintenance or renewal over the lifetime of the organism and to generate clonally related neural progeny. Neural stem cells give rise to neurons, astrocytes and oligodendrocytes during development and can replace a number of neural cells in the adult brain. Neural stem cells are neural cells for purposes of the present invention. The terms “neural cells” and “neuronal cells” are generally used interchangeably in many aspects of the present invention. Preferred neural cells for use in certain aspects according to the present invention include those cells which exhibit one or more of the neural/neuronal phenotypic markers such as Musashi-1, Nestin, NeuN, class III β-tubulin, GFAP, NF-L, NF-M, microtubule associated protein (MAP2), S100, CNPase, glypican (especially glypican 4), neuronal pentraxin II, neuronal PAS 1, neuronal growth associated protein 43, neurite outgrowth extension protein, vimentin, Hu, internexin, O4, myelin basic protein and pleiotrophin, among others.

The term “administration” or “administering” is used throughout the specification to describe the process by which cells of the subject invention, such as fetal neural stem cells obtained from umbilical cord blood, or more differentiated cells obtained therefrom, are delivered to a patient for therapeutic purposes. Cells of the subject invention be administered a number of ways including, but not limited to, parenteral (such term referring to intravenous and intra-arterial as well as other appropriate parenteral routes) and intrathecal administration, among others which term allows cells of the subject invention to migrate to the ultimate target site where needed. Cells of the subject invention can be administered in the form of intact CTX0E03 immortalized fetal neural stem cells. The compositions according to the present invention may be used without cell expansion, i.e. passaging, with a mobilization agent or differentiation agent. Administration will often depend upon the disease or condition treated and may preferably be via a parenteral route, for example, intravenously. In the case of stroke, the preferred route of administration will depend upon where the stroke is, but may be directly into the carotid artery, or may be administered systemically. In a preferred embodiment of the present invention, the route of administration for treating an individual post-stroke is systemic, via intravenous or intra-arterial administration. Optionally, the fetal neural stem cells are administered in conjunction with an immunosuppressive agent, such as cyclosporine A or tacrolimus.

The fetal neural stem cells of the present invention can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

The pharmaceutical compositions may further comprise a pharmaceutically acceptable carrier. Pharmaceutical compositions comprise an effective number of cells, optionally, in combination with a pharmaceutically-acceptable carrier, additive or excipient. In certain aspects of the present invention, cells are administered to the patient in need of a transplant in sterile saline. In other aspects of the present invention, the cells are administered in Hanks Balanced Salt Solution (HBSS) or Isolyte S, pH 7.4. Other approaches may also be used, including the use of serum free cellular media. Systemic administration of the cells to the patient may be preferred in certain indications, whereas direct administration at the site of or in proximity to the diseased and/or damaged tissue may be preferred in other indications.

In some embodiments, the CTX0E03 cells can be cryopreserved in a medium described by Hope, et al. (WO/2010/064054), in order to generate a frozen cell product that can be stably manufactured, stored and shipped to the treatment site, thawed and used without washing or further significant manipulation.

Pharmaceutical compositions according to the present invention preferably comprise an effective number within the range of about 1.0×10⁴ cells to about 1.0×10⁹ cells, more preferably about 1×10⁵ to about 1×10⁷ cells, even more preferably about 2×10⁵ to about 8×10⁶ cells generally in solution, optionally in combination with a pharmaceutically acceptable carrier, additive or excipient.

The term “non-tumorigenic” refers to the fact that the cells do not give rise to a neoplasm or tumor. Stem and/or progenitor cells for use in the present invention are preferably free from neoplasia and cancer.

Thus, fetal neural stem cells, or progenitor cells are the targets of gene transfer either prior to differentiation or after differentiation to a neural cell phenotype. The umbilical cord blood stem or progenitor cells of the present invention can be genetically modified with a heterologous nucleotide sequence and an operably linked promoter that drives expression of the heterologous nucleotide sequence. The nucleotide sequence can encode various proteins or peptides of interest. The gene products produced by the genetically modified cells can be harvested in vitro or the cells can be used as vehicles for in vivo delivery of the gene products (i.e., gene therapy).

The following written description provides exemplary methodology and guidance for carrying out many of the varying aspects of the present invention.

Standard molecular biology techniques known in the art and not specifically described are generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989). Polymerase chain reaction (PCR) is carried out generally as in PCR Protocols: A Guide to Methods and Applications, Academic Press, San Diego, Calif. (1990). Reactions and manipulations involving other nucleic acid techniques, unless stated otherwise, are performed as generally described in Sambrook et al., Molecular Cloning: A Laboratory Manual, Cold Springs Harbor Laboratory Press, and methodology as set forth in U.S. Pat. Nos. 4,666,828; 4,683,202; 4,801,531; 5,192,659; and 5,272,057 and incorporated herein by reference. In situ PCR in combination with Flow Cytometry can be used for detection of cells containing specific DNA and mRNA sequences (see, for example, Testoni et al., Blood, 1996, 87:3822).

Standard methods in immunology known in the art and not specifically described are generally followed as in Stites et al. (Eds.), Basic And Clinical Immunology, 8^{th} Ed., Appleton & Lange, Norwalk, Conn. (1994); and Mishell and Shigi (Eds.), Selected Methods in Cellular Immunology, W.H. Freeman and Co., New York (1980).

The CTX0E03 cells (ReNeuron L.td., Guildford, UK) were grown as previously described (Pollock K, et al. (2006). A conditionally immortal clonal stem cell line from human cortical neuroepithelium for the treatment of ischemic stroke. Exp Neurol. 199:143-155; Hodges H, et al. (2007). Making stem cell lines suitable for transplantation. Cell Transplant. 16:101-115). In brief, the cells were revived at passage 33 and plated onto laminin (Invitrogen, Carlsbad, Calif.) at a density of 2×10⁷ cells in 35 ml of media per T175 flask (Pollock K, et al. (2006). A conditionally immortal clonal stem cell line from human cortical neuroepithelium for the treatment of ischemic stroke. Exp Neurol. 199:143-155; Hodges H, et al. (2007). Making stem cell lines suitable for transplantation. Cell Transplant. 16:101-115). The cells were grown for 4 days or until ˜80% confluence before harvesting and resuspending in Hank's balanced salt soluton (Invitrogen) and N-acetylcycsteine (Sigma, St. Louis., Mo. vehicle) at a concentration of 5×10⁴ cells/μL.

All experiments were conducted in accordance with the National Institutes of Health guidelines, and were approved by the Institutional Animal Care and Use Committee of the University of South Florida, College of Medicine.

### EXAMPLE 1

Adult male Sprague Dawley (SD) rats (Harlan) weighing 225-250 g, were housed in a temperature controlled room with a 12 h light/dark cycle and given free access to food and water. A transient intraluminal occlusion stroke model as previously described by (Yasuhara T. et al. (2008). Intravenous grafts recapitulate the neurorestoration afforded by intracerebrally delivered multipotent adult progenitor cells in neonatal hypoxic-ischemic rats. J Cereb Blood Flow Metab. 28(11):1804-10. Epub 2008 Jul. 2) was used in this study. Rats were anesthetized with 5% isoflurane (3% maintenance) and a filament embolus was introduced into the right MCAO and secured in place for 1 hr. For the first 8 minutes, the left MCAO was ligated to reduce collateral reperfusion that could prevent the infarct. Laser doppler measurement of the cerebral blood flow was used to confirm lesioning with a drop of less than 70% being exclusion criteria. Animals were also excluded from the study retrospectively, if on post-mortem examination of the brain, considerable damage or scar tissue was observed, particularly cyst formation, or if the animal died before conclusion of the study, or showed unusual behavior, e.g. head tilt. One hour later, under anesthesia, the filament embolus was removed from the right MCAO and the incision sutured and the rat allowed to recover with appropriate post-operative survival procedures

Two days after MCAO, the animals were divided into two groups treated i.v. with either cells or vehicle. The animals were anesthetized with 5% isoflurane (3% maintenance) and the right jugular vein was exposed. Animals were randomly assigned to be injected with either 0.5 mls of vehicle (Hank's Balanced Salt Solution; HBSS+0.5 mM N-acetyl cysteine; NAC) or 1×10⁷ CTX0E03 cells in the same vehicle, over a 1 minute period. The incision was sutured and the rat allowed to recover with appropriate monitoring.

Cryopreserved CTX0E03 cells were thawed and plated on laminin-coated flasks in medium as described previously (Pollock, et al. (2006). A conditionally immortal clonal stem cell line from human cortical neuroepithelium for the treatment of ischemic stroke. Exp Neurol. 199(1)) and grown at 37° C., 5% CO₂ to 80% confluence before dissociation with TrypZean/EDTA [Cambrex] and Trituration solution (0.55 mg/ml Trypsin inhibitor [Sigma], 1% HSA, 25 U/ml Benzonase [Merck] in DMEM:F12) to neutralize the TrypZean and digest naked DNA. Following centrifugation and a wash in DMEM:F12 (Invitrogen), the cells were re-suspended in vehicle at a concentration of 2×10⁴ cells/ml.

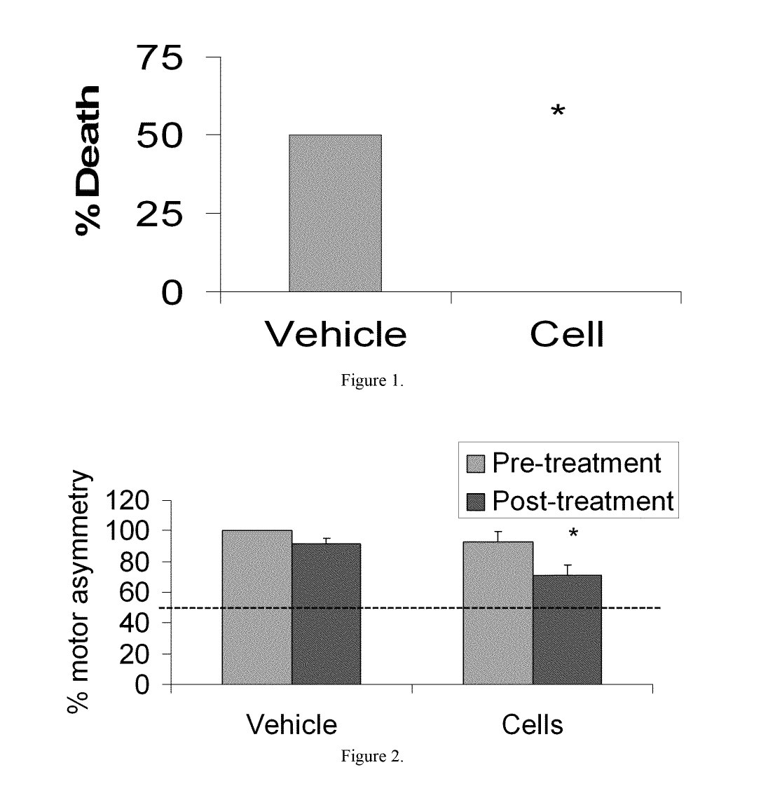

Four animals were injected with cells, whereas eight received vehicle. Three days after transplantation, half of the vehicle-treated animals had died compared with none of the cell-treated which was statistically significant as revealed by chi-squared analysis, as seen in FIG. 1. The motor asymmetry before and 3 days after transplant was found to be significantly reduced in the cell treated rats, seen in FIG. 2.

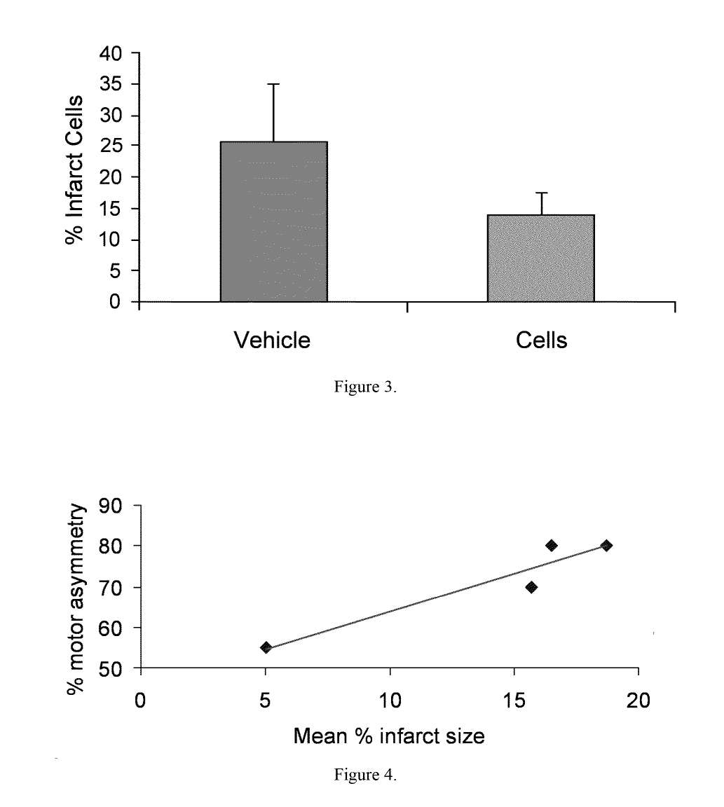

Three days after treatment, the rats were terminally anesthetized and perfused with cold saline. The brain was then removed and sliced into 2 mm coronal blocks. The blocks were then stained in 2% triphenyltetrazolium chloride (TTC) in PBS for 10 minutes in the dark. The brain slices were then fixed in 4% paraformaldehyde. The following day, six sections of the brain, covering the striatum, were photographed and the area of the infarct measured (lack of TTC staining) using ImageJ (NIH) by 2 observers blinded to the treatments. The infarct size was normalized to the contralateral hemisphere and calculated for the whole brain. Animal survival from treatment to perfusion between vehicle and cells was compared by chi-squared test. Infarct size was found not significantly different between cell and vehicle-treated rats, seen in FIG. 3. This may have been due to the sample size.

However, motor control testing did show a significant correlation between the % of motor asymmetry and the mean % infarct size in cell. Two days after MCAO, the surviving rats were behaviorally tested using the Elevated Body Swing Test (EBST) to determine motor asymmetry. The rat was held above bedding in a high-sided box by its tail and the direction the animal turns to is monitored 20 times. An unlesioned animal would be expected to turn left and right equally and therefore its motor asymmetry would be 50%. This was repeated three days after treatment by an individual blinded to the treatment and the values compared by t-test. As infarct size increased, the percent of asymmetry was found to increase in a linear relationship, as seen in FIG. 4. This result was not seen in vehicle-treated animals (data not shown).

There was therefore a significant improvement in motor behavior (as measured by EBST) and animal survival following cell transplant 2 days after MCAO. However, a similar significant change in infarct size was not observed, though there was a correlation with EBST (but not in the vehicle-treated animals), suggesting a significant difference may be present. The decreased mortality of animals treated with the CTX0E03 cells also suggests that not only is the transplantation of these cells safe, but that the cells also provide an improved outcome.

The i.v. implantation of CTX0E03 cells two days after experimental ischemic stroke exerts beneficial neurological effects. The grafted cells migrated to the injured site and either integrated with host cells or stimulated growth factor secretion to induce regenerative processes mediating the observed functional recovery.

### EXAMPLE 2

Twelve male 22-month old Fisher (F344) rats (NIA) were anesthetized with isoflurane and placed in a stereotaxic rig. Using a Hamilton syringe, either vehicle (n=6) or CTX0E03 cells (n=6; 4.5×10⁵ cells in 4.5 μl) were slowly implanted intracerebroventricularly at coordinates relative to bregma −1 mm anteriorly, +1.6 mm medially, and −4.5 mm dorsally to each rat. The following day, the rats were injected twice intraperitoneally with 50 mg/kg bromodeoxyuridine (5-bromo-2-deoxyoridine, BrdU; Sigma), 8 h apart, and were transcardially perfused with paraformaldehyde 1 day later. The brains were then removed and cryopreserved before being cut into 40 μm sagittal sections using a Microm cryostat (Richard-Allan Scientific, Kalamazoo, Mich.). Six animals from each group were implanted with either vehicle or cells.

Immunohistochemical staining for BrdU (marker of proliferation), doublecortin (DCX; immature neurons), ionized calcium-binding adaptor molecule I (IBA-1; microglia), glial fibrillary acidic protein (GFAP, astrocytes), and human nuclei antigen (HuNu; transplanted human fetal cortical cells) was performed on free floating sections as described previously (Bachstetter A D, et al. (2008). Peripheral injection of human umbilical cord blood stimulates neurogenesis in the aged rat brain. BMC Neurosci. 9:22). In brief, for BrdU staining, every sixth section of a series that surrounds the hippocampus were pretreated with 50% formaldehyde/2% SSC for 2 h at 65° C., followed by 30 min 2 N HCl at 37° C. and a borate buffer (pH 8.5) wash for antigen retrival. Endogenous peroxidase quenching in 0.3% hydrogen peroxide in methanol, followed by 1 h in blocking solution (3% normal horse serum and 0.25% Triton X-100 in 0.1 M PBS) were performed, followed by overnight incubation with mouse anti-rat BrdU (1:50; Roche, Indianapolis, Ind.). This was followed by a biotinylated secondary antibody (1:200; Vector laboratories, Burlingame, Calif.) and avidin-biotin substrate (ABU kit; Vector Laboratories) prior to diaminobenzidine substrate visualization. The sections were then mounted and coverslipped using Permount™ mounting medium (Fisher Chemicals, NJ). DCX can be used as a marker of migrating neurons, since it is expressed for ˜3 weeks from the creation of a new cell and has previously been shown to be a reliable indicator of neurogenesis (Rao M S & Shetty A K. (2004). Efficacy of doublecortin as a marker to analyse the absolute number and dendritic growth of newly generated neurons in the adult dentate gyms. Eur J Neurosci. 19:234-246; Couillard-Despres S, et al. (2005). Doublecortin expression levels in adult brain reflect neurogenesis. Eur J Neurosci. 21:1-14). DCX immunohistochemistry was performed without antigen retrival, using horse serum and a polyclonal goat antibody (1:150; Santa-Cruz Biotuch, CA) and the appropriate secondary antibody.

Immunofluorescence was used to compare colocalization of BrdU and IBA-1 or BrdU and GFAP and to demonstrate colocalization of BrdU and DCX. The 2 N HCl at room temperature was used for antigen retrival and primary incubation consisted of rat anti-BrdU (1:400; Accurate Chemical, Westbury, N.Y.) and the phenotype-defining primary antibodies [rabbit anti-GFAP (1:500; Dako, Carpinteria, Calif.), or rabbit anti-IBA1 (1:1,000; Wako, Richmond, Va.) or DCX (1:150; SantaCruz Biotech, CA)], overnight at 4° C. Visualization was achieved using the appropriate Alexafluor-conjugated secondary antibodies (Molecular Probes, CA) for 2 h and the sections were then mounted and coverslipped using Vectashield (Vector Labs). The presence of the transplanted cells was detected using the mouse monoclonal HuNu antibody (1:50; Chemicon, CA) that is specific for human nuclei. Visualization was achieved using an Alexafluor-conjugated secondary antibody (Molecular Probes). Quantification and imaging of labeled cells within the SGZ region was performed using the optical fractionator method of unbiased stereological cell counting (West M J, et al. (1991). Unbiased stereological estimation of the total number of neurons in the subdivisions of the rat hippocampus using the optical fractionator. Anat Rec. 231:482-497) using a Nikon Eclipse 600 (for BrdU+ cell) or Olympus BX 60 (for DCX+ cell) microscope and Stereo Investigator software (MicroBrightfield, VT). For the proliferation study, an identical virtual grid and counting frame of dimensions 125 μm×125 μm was used to enable us to count all the cells that were present in a section, due to the low number of BrdU+ cells observed in the aged animals. The anatomical structures were outlined using a 10×/0.45 objective, whereas a 60×/1.40 objective was used for cell quantification. For DCX cells, the virtual grid and counting frame were both 150 μm×150 μm. Outlines of the anatomical structures were done using a 10×/0.30 objective, whereas a 40×/0.75 objective was used for cell quantification. Defining the SGZ of the dentate gyms as a two-cell diameter band on both sides of the granular cell layer (GCL), the number of BrdU+ cells within the SGZ was counted. DCX+ cell counts were made in the SGZ/GCL, due to possible cell migration. To identify cell type-specific markers co-expressed in BrdU cells, immunofluorescent colocalization was assessed using an Olympus IX 70 microscope with a 10×/0.30, 20×/0.40 or 40×/0.60 objective and an Olympus DP 71 camera connected to a DP manager (Olympus, Japan). These cell counts were performed in the SGZ/GCL.

Data represent mean±SEM and statistical testing was by unpaired two-tailed t-test using P<0.05 as significant.

Twelve aged rats were implanted with either CTX0E03 cells or vehicle and treated with BrdU 24 h later. Forty-eight hours from the initial implant, the animals were perfused with paraformaldehyde, their brains removed and cryopreserved prior to sectioning sagitally at 40 μm. The sections were labeled with a number of different antibodies to determine cell proliferation, phenotype, and survival in the SGZ of the dentate gyms.

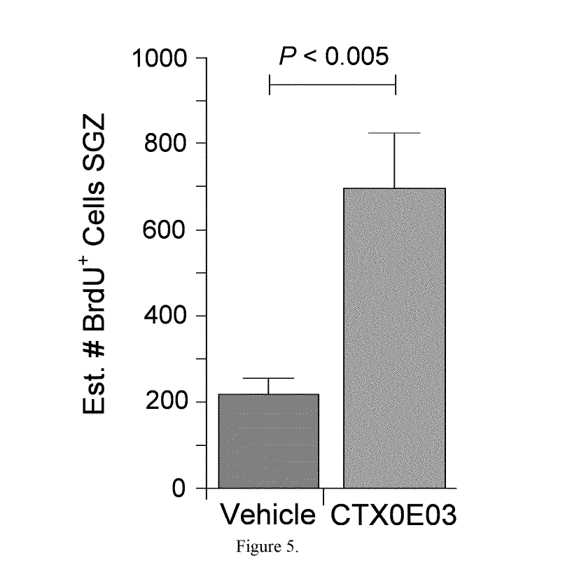

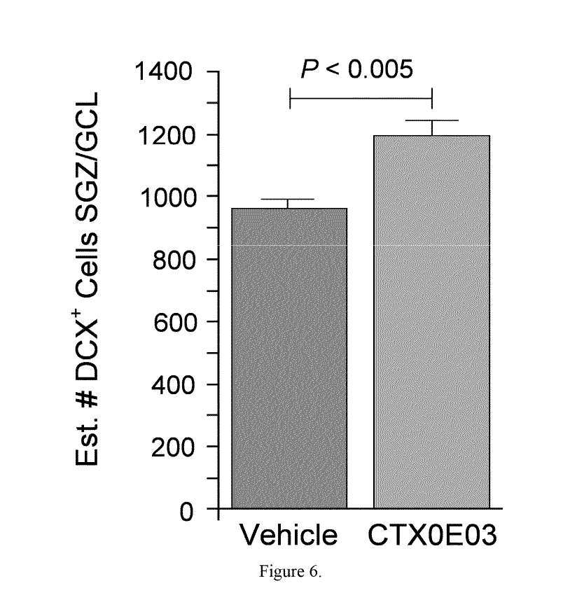

The presence of proliferating cells was determined using nuclear BrdU labeling. This was evident in the SGZ of the dentate gyms in both vehicle (218.0±31.00) and cell-treated (694.0±130.0) animals. A 3-fold significant increase in cell number was apparent in the cell-treated rats (t=3.894; df=9; P=0.0037; n=6), seen in FIG. 5. The presence of neuronal precursor cells was determined using DCX labeling of the SGZ. Labeled cells were seen in both vehicle (970±32.7) and cell-treated (1,202.4±61.9) animals, but again the number of cells was significantly increased in the cell-treated animals compared with the vehicle (t=4.29; df=8; P=0.002; n=5), as seen in FIG. 6.

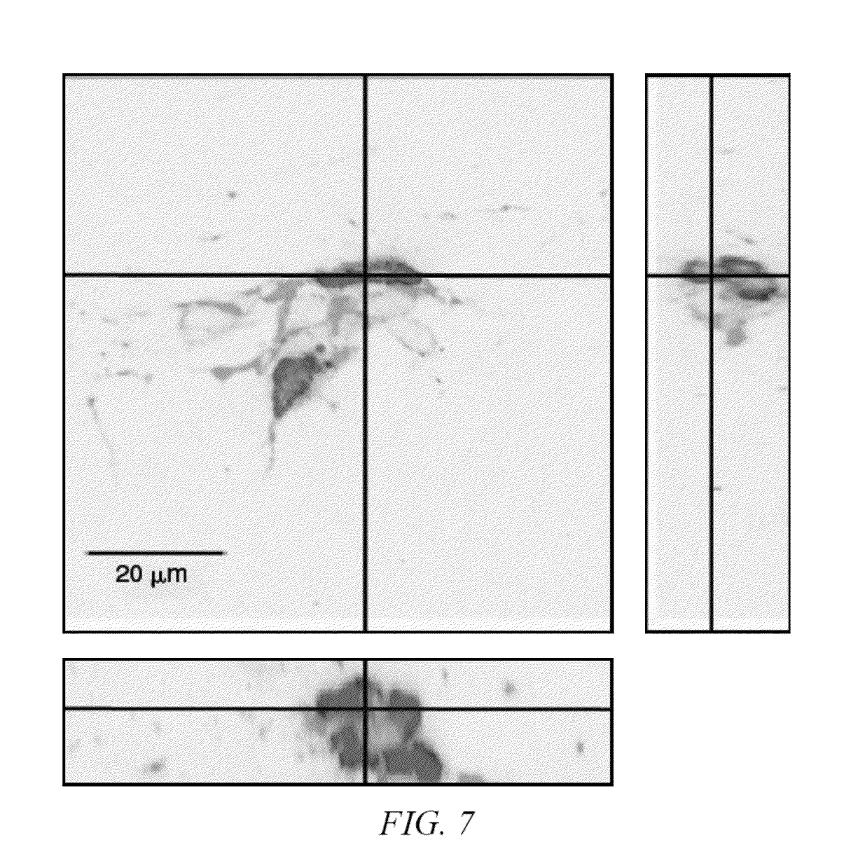

Confirmation that the DCX cells were also BrdU-positive was demonstrated by colocalization staining and confocal imaging, as seen in FIG. 7. Further identification of the potential phenotype of the proliferation cells was determined by using IBA-1 and GFAP staining for microglial and astrocytes, respectively, with the localization of BrdU.

Immunofluorescent IBA-1- and GFAP-positive staining cells were abundant, whereas nuclear BrdU-positive cells were rare. Thus, colocalization of BrdU and IBA-1 was very limited, and BrdU and GFAP co-expression was not found within the SGZ. No significant differences could be observed between staining in the vehicle- and cell-treated animals (data not shown).

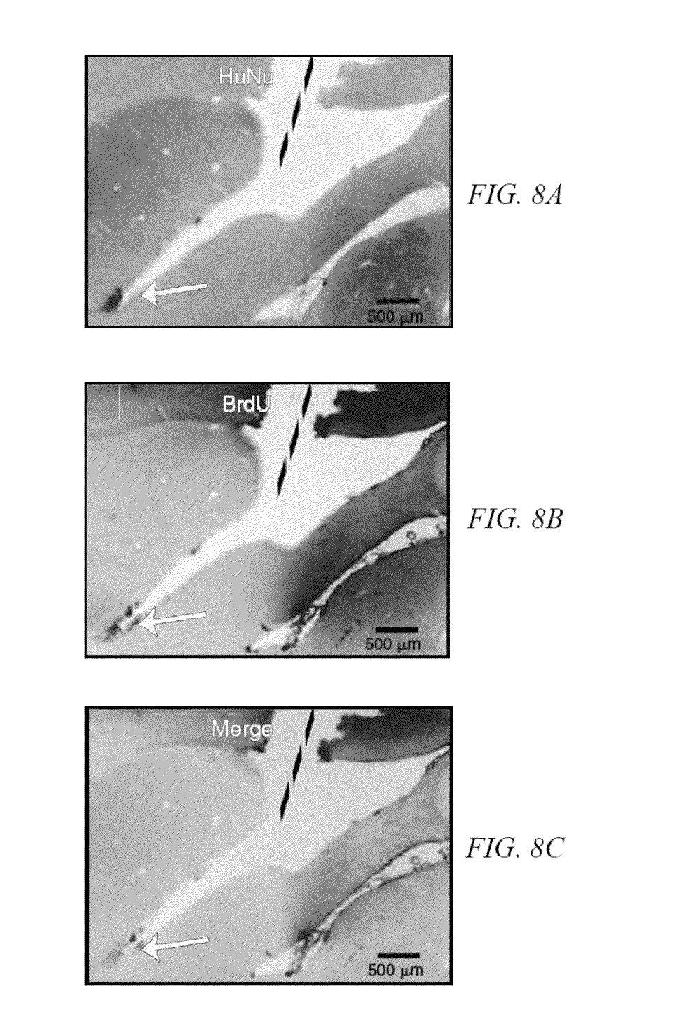

The presence of the transplanted cells at the injection site and in the SGZ was determined using HuNu staining. Human nuclei staining revealed no HuNu-positive cells within the SGZ, demonstrating that none of the BrdU-labeled cells were transplanted cells and instead were endogenous in origin. Some HuNu staining was apparent along the needle tract and in the ventricle, as seen in FIGS. 8(A) though (C). However, no HuNu-positive cells were found within the SGZ of either vehicle or cell-implanted rats, evidencing that none of the BrdU-labeled cells within the SGZ were transplanted cells, but instrade were endogenous in origin.

The absence of HuNu staining within the SGZ demonstrates that at 2 days from injection, the transplanted cells have not migrated to the region to either cause the effect or differentiate into immature neuronal cells, but instead are exerting their influence such as directly inducing cell proliferation or indirectly reducing inflammation to stimulate cell proliferation from the injection site. It is likely the cells are acting through the rapid secretion of anti-inflammatory cytokines, such as IL-10, or neurotrophic factors, such as brain-derived neurotrophic factor, nerve growth factor, or neurotrophin-3, which have been known to encourage the growth and differentiation of new neurons. CTX0E03 cells were previously shown to secrete VEGF and other factors in vitro (Eve D J, et al. (2008). Release of VEGF by ReN001 cortical stem cells. Cell Transplant. 17:464-465). Palmer et al. (Palmer T D, et al. (2000). Vascular niche for adult hippocampal neurogenesis. J Comp Neurol. 425:479-494) reported that in the adult rat SGZ, neurogenesis occurs in close proximity to blood vessels, where VEGF expression is high and angiogenesis is ongoing. Based on this and other evidence, Palmer et al. (Palmer T D, et al. (2000). Vascular niche for adult hippocampal neurogenesis. J Comp Neurol. 425:479-494) argued that neurogenesis and angiogenesis might be mechanistically linked, citing VEGF as a factor that might provide such a linkage. In addition, it has been shown that intracerebroventricular infusion of VEGF stimulated the proliferation of neuronal precursors in the SGZ and SVZ both in vitro and in vivo (Jin K, et al. (2002). Vascular endothelial growth factor (VEGF) stimulates neurogenesis in vitro and in vivo. Proc Natl Acad Sci USA. 99:11946-11950; Sun Y, et al. (2006). Vascular endothelial growth factor-B (VEGFB) stimulates neurogenesis: evidence from knockout mice and growth factor administration. Dev Biol. 289:329-335). Without being bound to any specific theory, given the above observations, the effects of CTX0E03 cells on endogenous neural proliferation may be modulated by VEGF. This could include the use of conditioned media in which the cells have secreted factors such as VEGF and attenuated cells for transplant, for example cells attenuated by freeze-thaw activity or heat inactivation. This would show that the effect is due to the factors secreted by the cells rather than the cells themselves.

Intracerebroventricular transplantation of CTX0E03 cells into rat brain results in increased proliferation within at least one of the endogenous stem cell reservoirs of the brain, the SGZ. This proliferation is of immature neuronal cells as shown by the increased DCX staining but the absence of significant IBA-1 and GFAP colocalization with BrdU. Confirmation that the neuronal precursors revealed by DCX staining were also proliferative (as shown by the BrdU colocalization) was also obtained.

While CTX0E03 cells do seem to have an effect on endogenous neuronal proliferation, it is not clear exactly how this occurs. Previously work has shown that reducing neuroinflammation in rats be blocking the conversion of pro-interleukin (IL)-1β to IL-1β through inhibition of the converting enzyme caspase-1 rescued some rats from age-related decreases in neurogenesis (Gemma C, et al. (2007) Blockade of caspase-1 increases neurogenesis in the aged hippocampus. Eur J Neurosci. 26:2795-2803) and resulted in an improvement in cognitive function, which is often affected by stroke related brain damage (Gemma C, et al. (2005). Improvement of memory for context by inhibition of caspase-1 in aged rats. Eur J Neurosci. 22:1751-1756). Furthermore, with hUCBCs, exogenous stem cells stimulate the endogenous neural progenitor cells to increase proliferation, and reduce neuroinflammation as evidenced by a decrease in the number of activated microglia (Bachstetter A D, et al. (2008). Peripheral injection of human umbilical cord blood stimulates neurogenesis in the aged rat brain. BMC Neurosci. 9:22). No significant increase in the negligible number of colocalized BrdU- and IBA-positive cells was observed between vehicle and cells at the site of proliferation, suggesting that neither the cells nor the injection had induced an immune response of new microglial cells. Further, previous work has shown that administration of human peripheral blood mononuclear cells as a control for the effect of human umbilical cord blood delivering cells did not alter neuronal proliferation or hippocampal neurogenesis (Bachstetter A D, et al. (2008). Peripheral injection of human umbilical cord blood stimulates neurogenesis in the aged rat brain. BMC Neurosci. 9:22). As well as the observed increased neuronal proliferation within the dentate gyms following hUCBC transplantation (Bachstetter A D, et al. (2008). Peripheral injection of human umbilical cord blood stimulates neurogenesis in the aged rat brain. BMC Neurosci. 9:22), glial restricted progenitors or NSCs from rats and mice have also been shown to promote endogenous NSCs number and survival in a more long-term study in younger rats (12 months compared with 22 months) and a 3-fold increase in cell number in the cell-transplanted animal. (Hattiangady B, et al. (2007). Increased dentate neurogenesis after grafting of glial restricted progenitors or neural stem cells in the aging hippocampus. Stem Cells. 25:2104-2117).

A clonal human NSC line, CTX0E03, has conditionally immortalized using the fusion transgene c-mycER^{TAM} to allow controlled expansion when cultured in the presence of 4-hydroxy-tamoxifen. No safety or toxicology issues identified in in vivo studies with this cell line. The data presented herein evidences an additional use of CTX0E03 cells to promote the endogenous restorative properties of the brain.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of an intravenous or intraarterial treatment for a neurodegenerative disease, it will be apparent to those skilled in the art that variations and modifications are possible without deviating from the broad spirit and principle of the present invention. It is intended that all matters contained in the foregoing description or shown in the accompanying drawings shall be interpreted as illustrative and not in a limiting sense. It is also to be understood that the following claims are intended to cover all of the generic and specific features of the invention herein described, and all statements of the scope of the invention which, as a matter of language, might be said to fall therebetween.

What is claimed is:

1. A method of treating a neurodegenerative disease or neurological injury in a patient, comprising:
administering a therapeutically effective amount of CTX0E03 cells to the patient,
wherein the administration is performed intravenously or intraarterially.

2. The method of claim 1, wherein the CTX0E03 cells are administered at about 1.0×10⁴ cells to about 1.0×10⁹ cells.

3. The method of claim 2, wherein the CTX0E03 cells are administered at about 1×10⁵ to about 1×10⁷ cells.

4. The method of claim 1, wherein 1×10⁷ CTX0E03 cells are administered.

5. The method of claim 1, further comprising administering the CTX0E03 cells in a therapeutic composition further comprising Hank's balanced salt solution and N-acetylcycsteine.

6. The method of claim 1, wherein the CTX0E03 cells are cryopreserved CTX0E03 cells.

7. The method of claim 6, wherein the CTX0E03 cells are passaged before administration into the patient.

8. The method of claim 1, wherein the neurodegenerative disease is stroke.

9. The method of claim 8, wherein the CTX0E03 cells are administered within the first 7 days of the stroke.

10. The method of claim 9, wherein the CTX0E03 cells are administered within 2 days of the stroke.

11. A method of treating a neurodegenerative disease in a patient, comprising:
administering 1×10⁷ CTX0E03 cells to the patient,
wherein the administration is performed intravenously or intraarterially.

12. The method of claim 11, further comprising administering the CTX0E03 cells in a therapeutic composition further comprising Hank's balanced salt solution and N-acetylcycsteine.

13. The method of claim 11, wherein the CTX0E03 cells are cryopreserved CTX0E03 cells.

14. The method of claim 13, wherein the CTX0E03 cells are passaged before administration into the patient.

15. The method of claim 11, wherein the neurodegenerative disease is stroke.

16. The method of claim 15, wherein the CTX0E03 cells are administered within 7 days of the stroke.

17. The method of claim 16, wherein the CTX0E03 cells are administered within 2 days of the stroke.

18. A method for repairing neural damage caused by a disease or disorder comprising
administering 1×10⁷ CTX0E03 cells to the patient,
wherein the administration is performed intravenously or intraarterially.

19. The method of claim 18, wherein the CTX0E03 cells are cryopreserved CTX0E03 cells.

20. The method of claim 19, wherein the CTX0E03 cells are passaged before administration into the patient.

21. The method of claim 18, wherein the neurodegenerative disease is stroke.

22. The method of claim 21, wherein the CTX0E03 cells are administered within 7 days of the stroke.

23. The method of claim 22, wherein the CTX0E03 cells are administered within 2 days of the stroke.
